(19) United States
(12) Patent Application Publication
(10) Pub. No.: US 2024/0408154 A1
(43) Pub. Date: Dec. 12, 2024

(54) ENHANCER OF NUMBER OF TCR CLONES

(71) Applicant: KABUSHIKI KAISHA YAKULT HONSHA, Tokyo (JP)

(72) Inventors: Ayaka MAKI, Tokyo (JP); Tomoaki NAITO, Tokyo (JP); Saya TAJIMA, Tokyo (JP); Tetsuji HORI, Tokyo (JP); Satoshi MATSUMOTO, Tokyo (JP)

(73) Assignee: KABUSHIKI KAISHA YAKULT HONSHA, Tokyo (JP)

(21) Appl. No.: 18/705,636

(22) PCT Filed: Oct. 28, 2022
(86) PCT No.: PCT/JP2022/040333

§ 371 Date: Apr. 29, 2024

(30) Foreign Application Priority Data
Nov. 2, 2021 (JP) 2021-179778

(51) Int. Cl. A61K 35/744; A23L 33/135; A61P 37/04; C12N 1/20

(52) U.S. Cl. CPC A61K 35/744; A23L 33/135; A61P 37/04; C12N 1/20

(57) ABSTRACT

Provided is an enhancer of number of TCR clones containing live lactic acid bacteria as an active ingredient. Provided is an enhancer of number of TCR clones, containing live lactic acid bacteria as an active ingredient, wherein after vaccination with CD4 carrying TRBV19 such as vaccination with IEV or a virus carrying an epitope recognized by CD8 positive T cells, the number of TCR clones carrying TRBV19 is enhanced.

### TECHNICAL FIELD

The present invention relates to an enhancer of number of clones of a T cell antigen receptor.

### BACKGROUND ART

A T cell antigen receptor (hereinafter, referred to as “TCR”) is an antigen receptor expressed on a cell membrane of a T cell, which is a type of lymphocyte. T cells are roughly divided into CD4 positive T cells and CD8 positive T cells, and a dimer of an α chain and a β chain or a γ chain and a δ chain is formed on the cell membrane by reconstituting a TCR gene in course of development. Variable regions of these dimers (hereinafter, referred to as a “V region”) are a main element of antigen recognition.

Non Patent Literature 1 reports that most of CD8 positive T cells specific for an epitope of a type-A influenza virus carry TRBV19, which is a specific gene fragment, on the β-chain V region of the TCR.

In addition, it is generally known that many immune cells are present in a gastrointestinal tract, and immune functions are enhanced by adjusting intestinal environment.

### CITATION LIST

### Non Patent Literature

Non Patent Literature 1: J Biol Chem. 2017 292 (45) 18618-18627

### SUMMARY OF INVENTION

### Technical Problem

However, there are many unclear points regarding the relationship between the intestinal environment and the number of TCR clones in a case where a vaccine against a virus is inoculated, and there remains room for research and development.

Therefore, an object of the present invention is to elucidate the relationship between live lactic acid bacteria carrying a function of adjusting the intestinal environment and the number of TCR clones after vaccination against a predetermined virus, and to provide an enhancer of number of TCR clones.

### Solution to Problem

As a result of intensive studies, the present inventors have surprisingly found that the number of TCR clones carrying TRBV19 is enhanced after vaccination with a predetermined virus by using live lactic acid bacteria belonging to the genus Lacticaseibacillus as an active ingredient. Here, the predetermined virus includes a virus carrying an epitope recognized by CD4 or CD8 positive T cells carrying TRBV19. The predetermined virus is not particularly limited as long as it satisfies the above conditions, and includes, for example, an influenza virus (hereinafter, referred to as “IFV”), a novel coronavirus (hereinafter, referred to as “COVID-19”), and the like. In addition, the vaccine of the predetermined virus includes, for example, an IFV vaccine, a COVID-19 vaccine, and the like. Characteristics of the enhancer of number of TCR clones based on this finding are as follows.

[1] An enhancer of number of TCR clones, containing live lactic acid bacteria belonging to the genus Lacticaseibacillus as an active ingredient, wherein after vaccination with CD4 carrying TRBV19 or a virus carrying an epitope recognized by CD8 positive T cells, the number of TCR clones carrying TRBV19 is enhanced.

[2] The enhancer of number of TCR clones according to [1], wherein the virus is an influenza virus or a novel coronavirus.

[3] The enhancer of number of TCR clones according to [1] or [2], wherein the lactic acid bacteria are classified into Lacticaseibacillus paracasei.

[4] The enhancer of number of TCR clones according to any one of [1] to [3], wherein the lactic acid bacteria are Lacticaseibacillus paracasei YIT 9029.

[5] The enhancer of number of TCR clones according to any one of [1] to [4], wherein the enhancer enhances the number of TCR clones carrying any one or more of TRBV6-2, TRBV9, TRBV10-3, or TRBV12-4 in addition to the TRBV19.

[6] The enhancer of number of TCR clones according to any one of [1] to [5], wherein live lactic acid bacteria are ingested 2.0×10 to the power of 10 or more per day.

[7] A food product for enhancing number of TCR clones, containing an enhancer of number of TCR clones according to any one of [1] to [6].

### Advantageous Effects of Invention

According to the present invention, it is possible to provide an enhancer of number of TCR clones, which enhances the number of TCR clones carrying TRBV19 after vaccination with the predetermined virus, using live lactic acid bacteria as an active ingredient. Moreover, as described above, the predetermined virus includes a virus carrying an epitope recognized by CD4 or CD8 positive T cells carrying TRBV19.

### BRIEF DESCRIPTION OF DRAWINGS

FIG. 1 is a diagram showing transition of the number of TCR clones carrying TRBV19 in a group receiving live Lacticaseibacillus paracasei YIT 9029 (LcS), a dead LcS ingestion group, and a non-drinking group.

FIG. 2 is a diagram showing transition of number of TCR clones carrying TRBV6-2, TRBV9, TRBV10-3, TRBV12-4, and TRBV19 for an IFV vaccinated group.

### DESCRIPTION OF EMBODIMENTS

Hereinafter, an enhancer of number of TCR clones using the lactic acid bacteria of the present invention as an active ingredient will be described in detail, but description of constituent elements described below is an example as one embodiment of the present invention and is not limited to these contents.

The enhancer of number of TCR clones of the present invention (hereinafter, it may be simply referred to as an “enhancer”) contains live lactic acid bacteria belonging to the genus Lacticaseibacillus as an active ingredient. In addition, the enhancer of number of TCR clones is characterized by enhancing the number of TCR clones carrying TRBV19, and further enhancing the number of TCR clones carrying, for example, any one or more of TRBV6-2, TRBV9, TRBV10-3, or TRBV12-4.

The lactic acid bacteria of the present invention include, for example, lactic acid bacteria belonging to the genus Lacticaseibacillus, and preferably include lactic acid bacteria belonging to Lacticaseibacillus paracasei. Although not particularly limited, for example, Lacticaseibacillus paracasei YIT 9029 (old classification: Lactobacillus casei YIT 9029) (FERM BP-1366) is preferable. The lactic acid bacteria may be used alone or in combination of two or more kinds thereof. Moreover, as described in the journal published in April 2020, lactic acid bacteria conventionally belonging to the genus Lactobacillus were subdivided, and the genus name was changed in some of the bacterial species.

### (Literature Related to Reclassification of Lactic Acid Bacteria)

Zheng et al., “A taxonomic note on the genus Lactobacillus: Description of 23 novel genera, emended description of the genus Lactobacillus Beijerinck 1901, and union of Lactobacillus and Leuconostocaceae.” Int. J. Syst. Evol. Microbiol. 2020 April; 70 (4): 2782-2858 DOI 10.1099/ijsem.0.004107

In the present description, it is assumed that the reclassification and subsequent new classifications are indicated. In addition, among the lactic acid bacteria classified into Lactobacillus casei or Lactobacillus paracasei under the old classification, lactic acid bacteria that can be newly classified as Lacticaseibacillus paracasei are included in Lacticaseibacillus paracasei of the present application.

The lactic acid bacteria include live bacteria and dead bacteria, and the live bacteria are used as the lactic acid bacteria as an active ingredient in the present invention. As an expected effect of the live bacteria (bodies) on lactic acid bacteria, there is an effect (probiotic) by remaining as the live bacteria in an intestinal tract and acting on intestinal bacterial flora. Moreover, as the effect of dead bacteria of lactic acid bacteria, an effect that a bacterial cell component or a metabolite directly acts on a living body is considered. Details of novel effects brought about by the live bacteria of lactic acid bacteria will be described in the examples.

A lower limit of the number of live lactic acid bacteria ingested per day in the enhancer of the present invention is preferably 2.0×10 to the power of 10 or more. More preferably, it is preferable to ingest 3.5×10 to the power of 10 or more.

An upper limit of the number of live lactic acid bacteria ingested per day in the enhancer of the present invention is preferably 1.0×10 to the power of 12 or less. More preferably, it is preferable to ingest 5.0×10 to the power of 11 or less. More preferably, it is preferable to ingest 1.1×10 to the power of 11 or less.

In a case where the number of bacteria ingested per day is within the above numerical range, the enhancer of the present invention significantly exhibits a function as an enhancer of the number of TCR clones. However, when the number of bacteria ingested per day is below the lower limit, the effect as the enhancer of number of TCR clones decreases. In addition, when the number of bacteria ingested per day is over the upper limit, the effect as the enhancer of number of TCR clones may reach a plateau.

The number of TCR clones is a value obtained by calculating the number of T cell clones carrying each TRBV detected from a sample based on specific V gene fragment (TRBV) information. For example, the number of TCR clones carrying TRBV19 is a value obtained by calculating the number of T cell clones carrying TRBV19 detected from a sample based on the V gene fragment information capable of specifying TRBV19.

The enhancer of number of TCR clones has the meaning of an agent that increases the number of T cell clones carrying a specific TRBV via gene rearrangement, or an agent that diversifies the types of TRBV expressed in T cells or that diversifies antigen recognition.

TRBV19 is known as a V gene fragment specific for an epitope of IFV, as described above. In addition, TRBV19 is also known as one of V gene fragments specific for an epitope of COVID-19. The vaccine of a virus carrying an epitope recognized by CD4 or CD8 positive T cells carrying TRBV19 of the present invention is not particularly limited as long as it is a virus carrying an epitope recognized by CD4 or CD8 positive T cells carrying TRBV19, and examples thereof include the IFV vaccine and the COVID-19 vaccine. Moreover, regarding COVID-19, it is pointed out that TRBV9 may also contribute to immunity. On the other hand, TRBV6-2 is known as a V gene fragment specific for an epitope related to chronic hepatitis B, HIV, and parasitic infection. TRBV12-4 is known to contribute to immunity against H5N6 avian influenza and herpes simplex.

Immunization produces antibodies in the body. Additionally, ingestion of live Lacticaseibacillus paracasei enhanced the number of TCR clones carrying TRBV19 after IFV vaccination. Since TRBV19 is one of V gene fragments specific to an epitope of IFV, if TRBV19 is a virus that specifically acts on an epitope similarly to IFV, it is highly probable that a similar effect (an increase in the number of TCR clones carrying TRBV19) can be obtained.

Therefore, in a case where TCR clones carrying a TRBV other than TRBV19 are expressed after inoculation of the IFV vaccine or the like by taking the enhancer of the present invention, it can be determined that the TCR clones are diversified (a TCR repertoire is enhanced). The enhancer of the present invention enhances the number of TCR clones carrying TRBV19, but can further enhance the number of TCR clones carrying any one or more of TRBV6-2, TRBV9, TRBV10-3, or TRBV12-4, and in particular, can enhance the number of TCR clones carrying TRBV6-2, TRBV9, TRBV10-3, and TRBV12-4.

In addition, when the increase in the number of TCR clones carrying TRBV19 is, for example, IFV, not only an influenza virus of a type contained in an inactivated IFV vaccine but also an infection protective effect on IFV of other types (a subtype not contained in the IFV vaccine) is expected. Therefore, even in a case where the number of T cell clones carrying TRBV19 is increased after IFV vaccination or the like as compared with a case where the enhancer of the present invention is ingested and a case where the enhancer is not ingested, it can be determined that the number of TCR clones is enhanced or the TCR repertoire is enhanced.

The enhancer of the present invention can take any form as long as it is suitable for ingestion. Examples thereof include but are not limited to, a pharmaceutical product, a quasi-pharmaceutical product, a health functional food, a specified health food, a nutritional functional food, a general food, a health supplement, a health food, a supplement, an enteral nutrient, an oral cosmetic, and a feed.

The enhancer of the present invention can be in the form of food product itself containing lactic acid bacteria from a viewpoint of palatability. Moreover, the food product includes a beverage. In particular, the food product is preferably a fermented milk food product, and the fermented milk food product includes not only a fermented milk specified by Ministerial Ordinance concerning Milk, but also a beverage containing live bacteria such as a dairy product lactic acid bacteria beverage and a lactic acid bacteria beverage, kefir, and yogurt. In addition, examples of the form include a hard type, a soft type, a plain type, a sweet type, a fruit type, a drink type, and a frozen type. In addition, from a viewpoint of storage stability, it can be in the form of granules, tablet pharmaceuticals, tablets, capsules, and the like.

In addition, in the food product of the present invention, other various food materials such as various carbohydrates, thickeners, emulsifiers, and various vitamins can be blended as necessary.

The enhancer of the present invention may indicate a description of its use, efficacy, function, ingestion method, and the like for a portion of the product obtained by packaging the enhancer. The “indicate” can be an indication suitable for a pharmaceutical product, a quasi-pharmaceutical product, a health functional food, a specified health food, a nutritional functional food, a general food, a health supplement, a health food, a supplement, an enteral nutrient, an oral cosmetic, and a feed.

The “indicate” includes all indications for informing a consumer of the above description, and includes all indications regardless of a purpose of indication, a content of indication, an object to be indicated, a medium to be indicated, and the like as long as the indication can recall and analogize the above indication content. For example, the above description may be indicated on a packaging or container of a product, the above description may be displayed or distributed in an advertisement, a price list, or a transaction document related to a product, or information containing the above description may be provided by a method via a telecommunication line (such as the Internet).

### EXAMPLES

### First Example

<Effect on Type of TCR Clones in a Case where Enhancer Containing Live Lactic Acid Bacteria as Active Ingredient is Ingested>

In First Example, the influence of live lactic acid bacteria on the TCR repertoire was analyzed.

Yakult 400 (registered trademark, manufactured by Kabushiki kaisha Yakult Honsha) was used as a beverage containing live lactic acid bacteria. Yakult 400 contains 40 billion or more live bacteria of Lacticaseibacillus paracasei YIT 9029 (LcS). First, 25 healthy adults were divided into 3 groups, Group 1 with 10 people who ingested one dose of Yakult 400 per day (hereinafter, the group is referred to as a “live LcS ingestion group”), Group 2 with 10 people who ingested one dose of heat-treated Yakult 400 per day (hereinafter, the group is referred to as a “dead LcS ingestion group”), and Group 3 with 5 people who did not ingest LcS (hereinafter, the group is referred to as a “non-drinking group”). Blood samples were collected at the start of drinking and two months after the start of drinking. In addition, all subjects received influenza virus vaccination (combination vaccine for 2018/2019:4 types in total of 2 strains of A and 2 strains of B) 3 weeks after the start of drinking.

Using the obtained blood samples, TCR repertoire analysis was performed in the following method. Blood was collected in a PAXgene (registered trademark) RNA blood collection tube and RNA was extracted. DNA was synthesized using human TCR β-chain-specific primers. Variable region genes including a complementarity determining region 3 (CDR3) were amplified by Multiplex PCR using 27 kinds of Forward primers binding to the V region of the human TCR B chain and one kind of Reverse primer binding to a constant region. Gene libraries were prepared from the obtained PCR product, and fastq files were acquired using a next-generation sequencer Miseq.

For data analysis, pRESTO was used as software, and IgBLAST (ftp://ftp.ncbi.nih.gov/blast/executables/igblast/release/) was used as a database. The analysis was performed on Linux command, and a base sequence of the complementarity determining region 3 (CDR3) and the V gene fragment (TRBV) information of the TCR β chain were acquired for each TCR clone. Subsequently, data analysis using RStudio was performed. For the analysis in RStudio, vegan and dplyr packages were used. Number of reads obtained from each sample was standardized at 12000, and non-productive TCR clones were excluded from the analysis. The number of TCR clones carrying TRBV19 was calculated based on the TRBV information of the TCR clones.

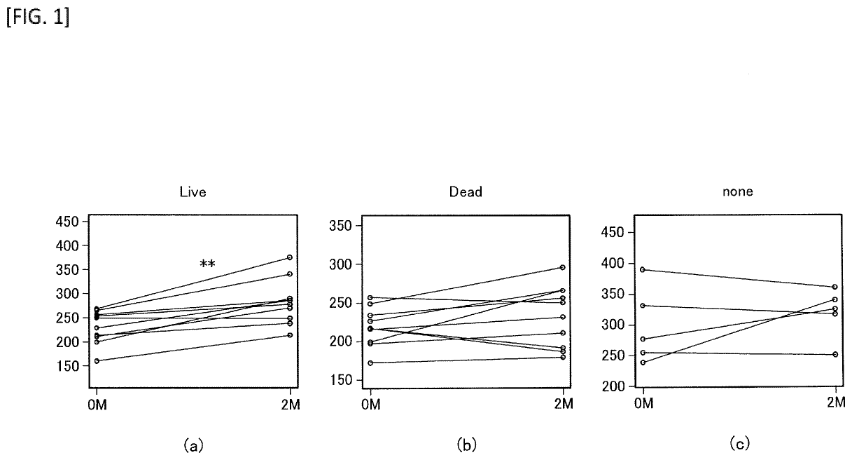

For each subject, the number of TCR clones with TRBV19 before the start of drinking and two months after the drinking was calculated. EZR (version 1.52) was used for statistical analysis and graph drawing. For each group, a Wilcoxon signed rank sum test was performed for comparative analysis before the start of drinking and two months after the drinking in each subject. The analysis results are shown in FIG. 1.

FIG. 1 shows the number of TCR clones in CD4 or CD8 positive T cells carrying TRBV19 before the start of drinking and after two months of drinking for the live LcS ingestion group, the dead LCS ingestion group, and the non-drinking group. Three graphs shown in FIG. 1 show, from left to right, the live LCS ingestion group (Live, a), the dead LcS ingestion group (Dead, b), and the non-drinking group (none, c). In each graph, a horizontal axis represents number of months elapsed (an alphabet M indicates number of months), and a vertical axis represents a value obtained by calculating the number of TCR clones carrying TRBV19. Each plot in the graph shows one subject. In addition, “**” indicates that a P value <0.01.

From FIG. 1, it was found that the number of TCR clones carrying TRBV19 two months after the drinking tended to significantly increase only in the live LCS ingestion group (Live) after IFV vaccination. Therefore, it was shown that the number of TCR clones carrying TRBV19 was enhanced or the TCR repertoire was enhanced only in a case where the live LcS ingestion group was used as an active ingredient.

### Second Example

### <Effects of IFV Vaccination and Ingestion of Predetermined Amount of Live LCS on TCR Clones>

In Second Example, the influence on the TCR repertoire in a case where a predetermined amount of live lactic acid bacteria was ingested was analyzed.

Peripheral blood was collected from 5 subjects (hereinafter, the group is referred to as a “non-vaccinated group”) who were not vaccinated with the influenza virus (IFV) combination vaccine (combination vaccine for 2019/2020:2 strains of A and 2 strains of B, in total 4 strains) and 5 subjects (hereinafter, the group is referred to as a “vaccinated group”) who were vaccinated with the vaccine among the healthy adults at the start of the test and two months later. In addition, the vaccinated group was vaccinated within 2 weeks after the first blood collection. The subjects recorded daily their intake of LcS-containing food product over the entire study period.

The peripheral blood was collected in a blood collection tube containing heparin. Using RosetteSep™ Human CD4⁺ T Cell Enrichment Cocktail (STEMCELL #15062) or RosetteSep™ Human CD8⁺ T Cell Enrichment Cocktail (STEMCELL #15063), CD4 positive T cells or CD8 positive T cells were recovered according to a protocol attached to the product.

RNA was extracted from the obtained cells, and cDNA was synthesized using human TCR β-chain-specific primers. Variable region genes including a complementarity determining region 3 (CDR3) were amplified by Multiplex PCR using 27 kinds of Forward primers binding to the V region of the human TCR B chain and one kind of Reverse primer binding to a constant region. The gene libraries were prepared and the fastq files were acquired using the next-generation sequencer Miseq.

pRESTO and IgBLAST were used for the data analysis. The fastq files were processed with Linux command to obtain the base sequence of the complementarity determining region 3 (CDR3) and the V gene fragment (TRBV) information of TCR3 chain. Subsequently, analysis using RStudio was performed, and the number of TCR clones was calculated as an α-diversity index of the TCR repertoire. Based on the V gene fragment (TRBV) information of TCR clones, the number of TCR clones carrying each TRBV was calculated.

EZR was used for statistical analysis and graph drawing. The Wilcoxon signed rank sum test was used for analysis of a before/after comparison in each subject.

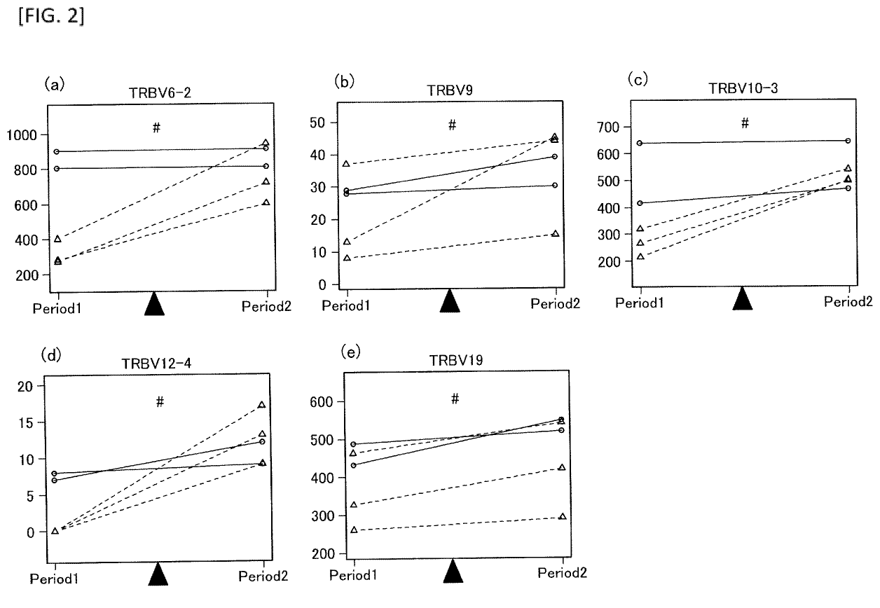

A change in the number of TCR clones with TRBV6-2, TRBV9, TRBV10-3, TRBV12-4, TRBV19 in CD4 positive T cells from the start of the test to two months for 5 of the vaccinated group is shown in FIG. 2. In FIG. 2, the horizontal axis of each graph represents time elapsed relative to blood collection of peripheral blood. Period 1 indicates blood collection at the start of the test, and Period 2 indicates blood collection after two months. The vertical axis indicates the number of TCR clones with each TRBV. In addition, “▴” on the horizontal axis indicates vaccination time. In addition, “#” indicates a p value <0.1.

In the graph, 3 subjects indicated by dotted lines were subjects who ingested 2.0×10 to the power of 10 or more live bacteria of LcS per day per week. In addition, 2 subjects indicated by a solid line were subjects who ingested less than 2.0×10 to the power of 10 live bacteria of LcS per day. Intake of live bacteria was calculated from the records of the subjects.

As a result, it was confirmed that the number of TCR clones in CD4 positive T cells was increased in the vaccinated group as compared with the non-vaccinated group. (Data not shown) In addition, from FIG. 2, increase rates of TRBV6-2, TRBV9, TRBV10-3, TRBV12-4, and TRBV19 were remarkable in the subjects who ingested 2.0×10 to the power of 10 or more live bacteria of LcS per day per week. Moreover, a utilization rate of TRBV19 was also increased in CD8 positive T cells (data is not shown). The “utilization rate” means a proportion of T cell clones carrying a specific TRBV gene among the T cell clones detected in the sample.

Therefore, the live lactic acid bacteria were found to be effective as an enhancer of the number of clones for enhancing the number of TCR clones carrying each TRBV after vaccination with IFV. In other words, it was shown to be effective as an enhancer of the TCR repertoire.

Furthermore, from these results, it was shown that even in a virus in which it is known that TRBV6-2, TRBV9, TRBV10-3, TRBV12-4, or TRBV19 are a V gene fragment specific to an epitope, for example, COVID-19 or the like, the number of TCR clones after vaccination could be enhanced by ingesting 2.0×10 to the power of 10 or more live bacteria of LcS per day per week.

In addition, it has become clear that the lower limit of the number of live lactic acid bacteria ingested per day is preferably 2.0×10 to the power of 10 or more, more preferably 3.5×10 to the power of 10 or more.

In addition, it has become clear that the upper limit to be ingested is preferably 1.0×10 to the power of 12 or less, more preferably 5.0×10 to the power of 11 or less, and still more preferably 1.1×10 to the power of 11 or less.

### Third Example

### <Analysis Method Regarding Novel Coronavirus (COVID-19) Vaccine>

In Third Example, an example of an analysis method regarding the COVID-19 vaccine is shown.

As a beverage containing live lactic acid bacteria, Yakult 400 (Manufactured by Yakult Honsha Co., Ltd., registered trademark) can be used.

Healthy adult subjects are divided into two groups, and the first group is a group (hereinafter, the group is referred to as a “live LcS ingestion group”) in which one dose of Yakult 400 is taken per day, and the second group is a group (hereinafter, the group is referred to as a “dead LcS ingestion group”) in which one dose of heat-treated Yakult 400 is taken per day.

Blood was collected at the start of drinking, two months after the start of drinking, or 4 months after the start of drinking.

The subjects were vaccinated with the coronavirus vaccine (COVID 19 Vaccine Moderna for intramuscular injection manufactured by Moderna, Inc., Comirnaty for intramuscular injection manufactured by Pfizer Corporation, or Vaxzevria for intramuscular injection manufactured by AstraZeneca K.K.) during the drinking period.

Using the obtained blood sample, the TCR repertoire analysis is performed in the following method. The blood was collected in a blood collection tube containing heparin, and peripheral blood mononuclear cells were fractionated by specific gravity centrifugation using a lymphocyte separation solution Lymphoprep. CD4 positive T cells or CD8 positive T cells were sorted using a cell sorter FACSAria, and RNA was extracted.

CDNA was synthesized using human TCR β-chain-specific primers.

Variable region genes including the complementarity determining region 3 (CDR3) were amplified by Multiplex PCR using 27 kinds of Forward primers binding to the V region of the human TCR β chain and one kind of Reverse primer binding to the constant region, the gene libraries were prepared from the obtained PCR product, and the fastq files were obtained using the next-generation sequencer Miseq.

The data analysis was performed in the same manner as in First Example.

The standardization of the number of reads in First Example was performed by changing from 12000 to any numerical value between 9000 and 10000.

The number of TCR clones with TRBV9, TRBV10-3, and TRBV19 before and after vaccination is calculated for each subject. The Wilcoxon signed rank sum test was performed for comparative analysis before the start of drinking and two months after the drinking in each subject.

For each subject, the increase rate in the number of TCR clones with TRBV9, TRBV10-3, and TRBV19 is calculated. A Mann-Whitney U test was performed for comparative analysis of a live LCS drinking group and a dead LcS drinking group.

The present application claims priority based on Japanese Patent Application No. 2021-179778 filed on Nov. 2, 2021, the entire contents of which are incorporated herein by reference.

1. An enhancer of number of TCR clones, comprising live lactic acid bacteria belonging to the genus Lacticaseibacillus as an active ingredient, wherein after vaccination with CD4 carrying TRBV19 or a virus carrying an epitope recognized by CD8 positive T cells, the number of TCR clones carrying TRBV19 is enhanced.

2. The enhancer of number of TCR clones according to claim 1, wherein the virus is an influenza virus or a novel coronavirus (COVID-19).

3. The enhancer of number of TCR clones according to claim 1, wherein the lactic acid bacteria are classified into Lacticaseibacillus paracasei.

4. The enhancer of number of TCR clones according to claim 1, wherein the lactic acid bacteria are Lacticaseibacillus paracasei YIT 9029.

5. The enhancer of number of TCR clones according to claim 1, wherein the enhancer enhances the number of TCR clones carrying any one or more of TRBV6-2, TRBV9, TRBV10-3, or TRBV12-4 in addition to the TRBV19.

6. The enhancer of number of TCR clones according to claim 1, wherein the live lactic acid bacteria are ingested 2.0×10 to the power of 10 or more per day.

7. A food product for enhancing number of TCR clones, comprising an enhancer of number of TCR clones according to claim 1.

8. The enhancer of number of TCR clones according to claim 1, wherein the virus is an influenza virus or a novel coronavirus (COVID-19), and wherein the lactic acid bacteria are classified into Lacticaseibacillus paracasei.

9. The enhancer of number of TCR clones according to claim 1, wherein the virus is an influenza virus or a novel coronavirus (COVID-19), and wherein the lactic acid bacteria are Lacticaseibacillus paracasei YIT 9029.

10. The enhancer of number of TCR clones according to claim 1, wherein the virus is an influenza virus or a novel coronavirus (COVID-19), and wherein the enhancer enhances the number of TCR clones carrying any one or more of TRBV6-2, TRBV9, TRBV10-3, or TRBV12-4 in addition to the TRBV19.

11. The enhancer of number of TCR clones according to claim 1, wherein the virus is an influenza virus or a novel coronavirus (COVID-19), and wherein the live lactic acid bacteria are ingested 2.0×10 to the power of 10 or more per day.

12. The enhancer of number of TCR clones according to claim 1, wherein the lactic acid bacteria are classified into Lacticaseibacillus paracasei, and wherein the enhancer enhances the number of TCR clones carrying any one or more of TRBV6-2, TRBV9, TRBV10-3, or TRBV12-4 in addition to the TRBV19.

13. The enhancer of number of TCR clones according to claim 1, wherein the lactic acid bacteria are classified into Lacticaseibacillus paracasei, and wherein the live lactic acid bacteria are ingested 2.0×10 to the power of 10 or more per day.

14. The enhancer of number of TCR clones according to claim 1, wherein the lactic acid bacteria are Lacticaseibacillus paracasei YIT 9029, and wherein the enhancer enhances the number of TCR clones carrying any one or more of TRBV6-2, TRBV9, TRBV10-3, or TRBV12-4 in addition to the TRBV19.

15. The enhancer of number of TCR clones according to claim 1, wherein the lactic acid bacteria are Lacticaseibacillus paracasei YIT 9029, and wherein the live lactic acid bacteria are ingested 2.0×10 to the power of 10 or more per day.

16. The enhancer of number of TCR clones according to claim 1, wherein the enhancer enhances the number of TCR clones carrying any one or more of TRBV6-2, TRBV9, TRBV10-3, or TRBV12-4 in addition to the TRBV19, and wherein the live lactic acid bacteria are ingested 2.0×10 to the power of 10 or more per day.

17. The enhancer of number of TCR clones according to claim 1, wherein the virus is an influenza virus or a novel coronavirus (COVID-19), wherein the lactic acid bacteria are Lacticaseibacillus paracasei YIT 9029, and wherein the enhancer enhances the number of TCR clones carrying any one or more of TRBV6-2, TRBV9, TRBV10-3, or TRBV12-4 in addition to the TRBV19.

18. The enhancer of number of TCR clones according to claim 1, wherein the virus is an influenza virus or a novel coronavirus (COVID-19), wherein the lactic acid bacteria are Lacticaseibacillus paracasei YIT 9029, and wherein the live lactic acid bacteria are ingested 2.0×10 to the power of 10 or more per day.

19. The food product for enhancing number of TCR clones according to claim 7, wherein the virus is an influenza virus or a novel coronavirus (COVID-19), wherein the lactic acid bacteria are Lacticaseibacillus paracasei YIT 9029.
